(19) United States
(12) Patent Application Publication
(10) Pub. No.: US 2006/0024331 A1
(43) Pub. Date: Feb. 2, 2006

(54) Toxin compounds with enhanced membrane translocation characteristics

(76) Inventors: Ester Fernandez-Salas, Fullerton, CA (US); Lance E. Steward, Irvine, CA (US); Wei-Jen Lin, Cerritos, CA (US); Kei Roger Aoki, Coto de Caza, CA (US); George Sachs, Encino, CA (US)

Correspondence Address: ALLERGAN, INC., LEGAL DEPARTMENT, 2525 DUPONT DRIVE, T2-7H, IRVINE, CA, 92612-1599, US

(21) Appl. No.: 10/909,769

(22) Filed: Aug. 2, 2004

(51) Int. Cl. A61K 39/08; C07K 14/33

(52) U.S. Cl. 424/239.1; 530/350

(57) ABSTRACT

The present invention relates to a compound comprising a toxin linked to a translocator. Non-limiting examples of toxins of the present invention are botulimum toxin, butyricum toxin, tetani toxins and the light chains thereof. In some embodiments, the translocator of the present invention comprises a protein transduction domain.

### FIELD OF INVENTION

This invention broadly relates to recombinant DNA technology. Particularly, the invention relates to toxin compounds linked to a translocator, wherein the translocator facilitates the translocation of the toxins across cell membranes.

### BACKGROUND

The genus Clostridium has more than one hundred and twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of foodborne botulism. The effects of botulism typically appear 18 to 36 hours after eating the foodstuffs contaminated with a Clostridium botulinum culture or spores. The botulinum toxin can apparently pass unattenuated through the lining of the gut and shows a high affinity for cholinergic motor neurons. Symptoms of botulinum toxin intoxication can progress from difficulty walking, swallowing, and speaking to paralysis of the respiratory muscles and death.

Botulinum toxin is the most lethal natural biological agent known to man. One mouse LD₅₀ unit of BOTOX® (purified neurotoxin complex, available from Allergan, Inc., of Irvine, Calif.) is about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the stated LD50 of botulinum toxin type A of 0.3 ng equals 1 U is corrected for the fact that about 0.05 ng of BOTOX® equals 1 unit). One unit (U) of botulinum toxin is defined as the LD50 upon intraperitoneal injection into female Swiss Webster mice weighing 18 to 20 grams each.

Seven generally immunologically distinct botulinum toxins have been characterized, these being respectively botulinum toxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD50 for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity receptors on cholinergic motor neurons, is translocated into the neuron and blocks the release of acetylcholine. Additional uptake can take place through low affinity receptors, as well as by phagocytosis and pinocytosis.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain (the H chain or HC), and a cell surface receptor. The receptor is thought to be different for each type of botulinum toxin and for tetanus toxin. The carboxyl end segment of the HC appears to be important for targeting of the botulinum toxin to the cell surface.

In the second step, the botulinum toxin crosses the plasma membrane of the target cell. The botulinum toxin is first engulfed by the cell through receptor-mediated endocytosis, and an endosome containing the botulinum toxin is formed. The catalytic LC then exits the endosome into the cytoplasm of the cell. This step is thought to be mediated by the amino end segment of the HC, the HN, that undergoes a conformational change in response to a pH of about 5.5 or lower. Endosomes are known to possess a proton pump which decreases intra-endosomal pH. The conformational shift exposes hydrophobic residues in the H_{N}, which permits the botulinum toxin to embed itself in the endosomal membrane forming a pore. The botulinum toxin (or at least the light chain of the botulinum) then translocates through the endosomal membrane into the cytoplasm.

The last step of the mechanism of botulinum toxin activity appears to involve reduction of the disulfide bond joining the heavy chain and the light chain. The entire toxic activity of botulinum and tetanus toxins is contained in the L chain of the toxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C1 was originally thought to cleave syntaxin, but was found to cleave both syntaxin and SNAP-25. Each of the botulinum toxins specifically cleaves a different bond, except botulinum toxin type B and tetanus toxin which cleave the same bond. Each of these cleavages block the process of vesicle-membrane docking, thereby preventing exocytosis of vesicle content.

Botulinum toxins have been used in clinical settings for the treatment of neuromuscular disorders characterized by hyperactive skeletal muscles (i.e. motor disorders). In 1989 a botulinum toxin type A complex was approved by the U.S. Food and Drug Administration for the treatment of blepharospasm, strabismus and hemifacial spasm. Subsequently, a botulinum toxin type A was also approved by the FDA for the treatment of cervical dystonia and for the treatment of glabellar lines, and a botulinum toxin type B was approved for the treatment of cervical dystonia. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

Although all the botulinum toxin serotypes apparently inhibit release of the neurotransmitter acetylcholine at the neuromuscular junction, they do so by affecting different neurosecretory proteins and/or cleaving these proteins at different sites as mentioned previously. For example, botulinum types A and E both cleave the 25 kiloDalton (kD) synaptosomal associated protein (SNAP-25), but they target different amino acid sequences within this protein.

Botulinum toxin types B, D, F and G act on vesicle-associated protein (VAMP, also called synaptobrevin), with each serotype cleaving the protein at a different site. Finally, botulinum toxin type C1 has been shown to cleave both syntaxin and SNAP-25. These differences in mechanism of action may affect the relative potency, tissue specificity, and/or duration of action of the various botulinum toxin serotypes. Apparently, a substrate for a botulinum toxin can be found in a variety of different cell types. See e.g. Biochem J 1;339 (pt 1):159-65:1999, and Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C1 is apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin and non-toxic nonhemaglutinin protein (NTNH) and/or non-toxin hemaglutinin proteins (HA) and a non-toxin and non-toxic nonhemaglutinin protein (NTNH). These non-toxin proteins (which along with the botulinum toxin molecule comprise the relevant neurotoxin complex) may act to provide stability against denaturation of the botulinum toxin molecule and protection against digestive acids and enzymes when a botulinum toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51(2);522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1897). Thus, when adequate concentrations are used, stimulus-evoked release of most neurotransmitters can be blocked by botulinum toxin. See e.g. Pearce, L. B., Pharmacologic Characterization of Botulinum Toxin For Basic Science and Medicine, Toxicon 35(9);1373-1412 at page 1393; Bigalke H., et al., Botulinum A Neurotoxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [3H]Noradrenaline and [3H]GABA From Rat Brain Homogenate, Experientia 44;224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented culture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C1, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on strains, the length of incubation, and the culture conditions. Therefore, a certain percentage of any preparation of, for example, the botulinum toxin type B toxin is likely to be inactive, possibly accounting in part for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection in human, a shorter duration of activity and is also less potent than botulinum toxin type A at the same dose level. High quality crystalline botulinum toxin type A can be produced from the Hall A strain of Clostridium botulinum with characteristics of ≧3×10⁷ U/mg, an A260/A278 of less than 0.60 and a distinct pattern of banding on gel electrophoresis. The known Schantz process can be used to obtain crystalline botulinum toxin type A, as set forth in Schantz, E. J., et al, Properties and use of Botulinum toxin and Other Microbial Neurotoxins in Medicine, Microbiol Rev. 56;80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD50 U/mg or greater; purified botulinum toxin type B with an approximately 156 kD molecular weight with a specific potency of 1-2×10⁸ LD50 U/mg or greater, and; purified botulinum toxin type F with an approximately 155 kD molecular weight with a specific potency of 1-2×10⁷ LD50 U/mg or greater.

Research-grade botulinum toxins and/or botulinum toxin complexes can be obtained from List Biological Laboratories, Inc., Campbell, Calif.; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical compound.

As with enzymes in general, the biological activity of the botulinum toxins (which are intracellular peptidases) is dependent, at least in part, upon their three dimensional conformation. Thus, botulinum toxin type A is inactivated by heat, various chemicals, surface stretching and surface drying. Additionally, it is known that dilution of a botulinum toxin complex obtained by the known culturing, fermentation and purification to the much, much lower toxin concentrations used for pharmaceutical compound formulation results in rapid inactivation of the toxin unless a suitable stabilizing agent is present. Dilution of the toxin from milligram quantities to a solution containing nanograms per milliliter presents significant difficulties because of the rapid loss of specific toxicity upon such great dilution. Since the botulinum toxin may be used months or years after the toxin containing pharmaceutical compound is formulated, the toxin is usually stabilized with a stabilizing agent such as albumin and gelatin.

A commercially available botulinum toxin containing pharmaceutical compound is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and associated NTNH and hemagglutinin proteins. The crystalline complex is re-dissolved in a solution containing saline and albumin and sterile filtered (0.2 microns) prior to vacuum-drying. The vacuum-dried product is stored in a freezer at or below-5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX®) can be stored in a refrigerator at about 2° C. to about 8° C. Reconstituted, refrigerated BOTOX® has been reported to retain its potency for at least about two weeks. Neurology, 48:249-53:1997.

It has been reported that botulinum toxin type A has been used in clinical settings as follows:
(1) about 75-125 units of BOTOX® per intramuscular injection (multiple muscles) to treat cervical dystonia;
(2) 5-10 units of BOTOX® per intramuscular injection to treat glabellar lines (brow furrows) (5 units injected intramuscularly into the procerus muscle and 10 units injected intramuscularly into each corrugator supercilii muscle);
(3) about 30-80 units of BOTOX® to treat constipation by intrasphincter injection of the puborectalis muscle;
(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.
(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).
(6) to treat upper limb spasticity following stroke by intramuscular injections of BOTOX® into five different upper limb flexor muscles, as follows:
   (a) flexor digitorum profundus: 7.5 U to 30 U
   (b) flexor digitorum sublimus: 7.5 U to 30 U
   (c) flexor carpi ulnaris: 10 U to 40 U
   (d) flexor carpi radialis: 15 U to 60 U
   (e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.
(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

It is known that botulinum toxin type A can have an efficacy for up to 12 months (European J. Neurology 6 (Supp 4): S111-S1150:1999), and in some circumstances for as long as 27 months, when used to treat glands, such as in the treatment of hyperhydrosis. See e.g. Bushara K., Botulinum toxin and rhinorrhea, Otolaryngol Head Neck Surg 1996;114(3):507, and The Laryngoscope 109:1344-1346:1999. However, the usual duration of an intramuscular injection of Botox® is typically about 3 to 4 months.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and Dysport® available from Beaufour Ipsen, Porton Down, England. A botulinum toxin type B preparation (MyoBloc®) is available from Elan Pharmaceuticals of San Francisco, Calif.

U.S. Pat. No. 5,989,545 discloses that a modified clostridial neurotoxin or fragment thereof, preferably a botulinum toxin, chemically conjugated or recombinantly fused to a particular targeting moiety can be used to treat pain by administration of the agent to the spinal cord. See also Cui et al., Subcutaneous administration of botulinum toxin A reduces formalin-induced pain, Pain, 2004 January; 107(1-2):125-133, the disclosure of which is incorporated in its entirety by reference herein.

It has been reported that use of a botulinum toxin to treat various spasmodic muscle conditions can result in reduced depression and anxiety, as the muscle spasm is reduced. Murry T., et al., Spasmodic dysphonia; emotional status and botulinum toxin treatment, Arch Otolaryngol 1994 March; 120(3): 310-316; Jahanshahi M., et al., Psychological functioning before and after treatment of torticollis with botulinum toxin, J Neurol Neurosurg Psychiatry 1992; 55(3): 229-231. Additionally, German patent application DE 101 50 415 A1 discusses intramuscular injection of a botulinum toxin to treat depression and related affective disorders.

A botulinum toxin has also been proposed for or has been used to treat skin wounds (U.S. Pat. No. 6,447,787), various autonomic nerve dysfunctions (U.S. Pat. No. 5,766,605), tension headache, (U.S. Pat. No. 6,458,365), migraine headache pain (U.S. Pat. No. 5,714,468), sinus headache (U.S. patent application Ser. No. 429069), post-operative pain and visceral pain (U.S. Pat. No. 6,464,986), neuralgia pain (U.S. patent application Ser. No. 630,587), hair growth and hair retention (U.S. Pat. No. 6,299,893), dental related ailments (U.S. provisional patent application Ser. No. 60/418,789), fibromyalgia (U.S. Pat. No. 6,623,742), various skin disorders (U.S. patent application Ser. No. 10/731,973), motion sickness (U.S. patent application Ser. No. 752,869), psoriasis and dermatitis (U.S. Pat. No. 5,670,484), injured muscles (U.S. Pat. No. 6,423,319) various cancers (U.S. Pat. No. 6,139,845), smooth muscle disorders (U.S. Pat. No. 5,437,291), down turned mouth corners (U.S. Pat. No. 6,358,917), nerve entrapment syndromes (U.S. patent application 2003 0224019), various impulse disorders (U.S. patent application Ser. No. 423,380), acne (WO 03/011333) and neurogenic inflammation (U.S. Pat. No. 6,063,768). Controlled release toxin implants are known (see e.g. U.S. Pat. Nos. 6,306,423 and 6,312,708) as is transdermal botulinum toxin administration (U.S. patent application Ser. No. 10/194,805).

Botulinum toxin type A has been used to treat epilepsia partialis continua, a type of focal motor epilepsy. Bhattacharya K., et al., Novel uses of botulinum toxin type A: two case reports, Mov Disord 2000; 15(Suppl 2):51-52.

It is known that a botulinum toxin can be used to: weaken the chewing or biting muscle of the mouth so that self inflicted wounds and resulting ulcers can heal (Payne M., et al, Botulinum toxin as a novel treatment for self mutilation in Lesch-Nyhan syndrome, Ann Neurol 2002 September;52(3 Supp 1):S157); permit healing of benign cystic lesions or tumors (Blugerman G., et al., Multiple eccrine hidrocystomas: A new therapeutic option with botulinum toxin, Dermatol Surg 2003 May;29(5):557-9); treat anal fissure (Jost W., Ten years' experience with botulinum toxin in anal fissure, Int J Colorectal Dis 2002 September;17(5):298-302, and; treat certain types of atopic dermatitis (Heckmann M., et al., Botulinum toxin type A injection in the treatment of lichen simplex: An open pilot study, J Am Acad Dermatol 2002 April;46(4):617-9).

Additionally, a botulinum toxin may have an effect to reduce induced inflammatory pain in a rat formalin model. Aoki K., et al, Mechanisms of the antinociceptive effect of subcutaneous Botox: Inhibition of peripheral and central nociceptive processing, Cephalalgia 2003 September;23(7):649; and Cui et al., Subcutaneous administration of botulinum toxin A reduces formalin-induced pain, Pain, 2004 January; 107(1-2):125-133, the disclosure of which is incorporated in its entirety by reference herein. Furthermore, it has been reported that botulinum toxin nerve blockage can cause a reduction of epidermal thickness. Li Y, et al., Sensory and motor denervation influences epidermal thickness in rat foot glabrous skin, Exp Neurol 1997; 147:452-462 (see page 459). Finally, it is known to administer a botulinum toxin to the foot to treat excessive foot sweating (Katsambas A., et al., Cutaneous diseases of the foot: Unapproved treatments, Clin Dermatol 2002 November-December;20(6):689-699; Sevim, S., et al., Botulinum toxin-A therapy forpalmar and plantar hyperhidrosis, Acta Neurol Belg 2002 December; 102(4):167-70), spastic toes (Suputtitada, A., Local botulinum toxin type A injections in the treatment of spastic toes, Am J Phys Med Rehabil 2002 October;81 (10):770-5), idiopathic toe walking (Tacks, L., et al., Idiopathic toe walking: Treatment with botulinum toxin A injection, Dev Med Child Neurol 2002;44(Suppl 91):6), and foot dystonia (Rogers J., et al., Injections of botulinum toxin A in foot dystonia, Neurology 1993 April;43(4 Suppl 2)).

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for ganglioside receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Neurotoxin Type A and Comparison with Other Clostridial Neurotoxins, J Biological Chemistry 265(16);9153-9158:1990.

Acetylcholine

Typically only a single type of small molecule neurotransmitter is released by each type of neuron in the mammalian nervous system, although there is evidence which suggests that several neuromodulators can be released by the same neuron. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the bag 1 fibers of the muscle spindle fiber, by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

Although botulinum toxin is successfully used for many indications, the use of botulinum toxin for the treatment of some diseases remain difficult due to the inability to deliver an effective dose of the toxin into targeted cells, since these cells do not possess high affinity uptake and/or the toxin receptors on the cell remain uncharacterized—for example, non-neuronal cells such as pancreatic cells. Thus, there remains a need for improved toxin compounds with enhanced cell membrane translocation characteristics.

### SUMMARY OF THE INVENTION

The present invention provides for that need. In accordance with the present invention, a compound is featured comprising a toxin linked to a translocator. Non-limiting examples of toxins of the present invention are botulinum toxin, butyricum toxin, tetani toxins and the light chains thereof. In some embodiments, the toxin comprises a light chain of a botulinum toxin type A, B, C₁, D, E, F, G, or mutated recombinant LCs with improved characteristics, or mixtures thereof. In some embodiments, the toxin comprises a light chain of a botulinum toxin type A, B, C₁, D, E, F or G, and a whole or part of a heavy chain of a botulinum toxin type A, B, C₁, D, E, F or G.

The translocator of the present invention provides for enhanced translocation of the toxin into cells. In some embodiments, the translocator comprises a protein transduction domain (PTD). Non-limiting examples of translocators include a ciliary neurotrophic factor, caveolin, interleukin 1 beta, thioredoxin, fibroblast growth factor-1, fibroblast growth factor-2, Human beta-3, integrin, lactoferrin, Engrailed, Hoxa-5, Hoxb-4, or Hoxc-8. Non-limiting examples of PTD include penetratin peptide, Kaposi fibroblast growth factor membrane-translocating sequence, nuclear localization signal, transportan, herpes simplex virus type 1 protein 22, and human immunodeficiency virus transactivator protein. In some embodiments, a compound of the present invention further comprises a protease cleavage domain and/or a targeting moiety.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

Definitions

“Light chain” (L chain, LC, or L) has a molecular weight of about 50 kDa. A light chain has proteolytic/toxic activity.

“Heavy chain” (H chain or H) has a molecular weight of about 100 kDa. A heavy chain comprises an H_{c} and an H_{N}.

“H_{c}” is the carboxyl end fragment of the H chain, which is involved in binding to cell surfaces.

“H_{N}” is the amino end segment of the H chain, which is involved in the translocation of at least the L chain across an intracellular endosomal membrane into a cytoplasm of a cell.

“Targeting moiety” means a chemical compound or peptide which is able to preferentially bind to a cell surface receptor under physiological conditions.

“Linked” in the context of one component of the invention (e.g., a toxin) being “linked” to other components of the invention (e.g., a translocator, a targeting moiety, etc.) means that the components may be linked via a covalent bond, a linker and/or a spacer.

“Linker” means a molecule which couples two or more other molecules or components together.

“Spacer” means a molecule or set of molecules which physically separate and add distance between the components. One function of a spacer is to prevent steric hindrance between the components. For example, an compound of the present invention may be: L-linker-spacer-linker-HN-linker-targeting moiety.

“About” means approximately or nearly and in the context of a numerical value or range set forth herein means ±10% of the numerical value or range recited or claimed.

“Locally administering” means direct administration of a pharmaceutical at or to the vicinity of a site on or within an animal body, at which site a biological effect of the pharmaceutical is desired. Local administration excludes systemic routes of administration, such as intravenous or oral administration.

### DESCRIPTION OF EMBODIMENTS

The present invention relates to compounds comprising a toxin linked to a translocator. The translocator of the present invention is a protein or a peptide or a peptidomimetic that facilitates the transport of the toxin across a cell membrane. In some embodiments, the translocator of the present invention functions independently of transporters or specific receptors. In some embodiments, the translocators of the present invention is not energy dependent. Without wishing to limit the invention to any theory or mechanism of operation, it is believed that the translocator comprises a PTD. Further, it is believed that the PTD is primarily responsible for the translocation of the toxin across a cell membrane. PTDs are amino acid sequence domains that have been shown to cross biological membranes efficiently and independently of transporters or specific receptors. See Moris M C et al., Nature Biotechnology, 19:1173-1176, the disclosure of which is incorporated in its entirety by reference herein.

In some embodiments, the translocator is a ciliary neurotrophic factor, caveolin, interleukin 1 beta, thioredoxin, fibroblast growth factor-1, fibroblast growth factor-2, Knotted-1, Human beta-3 integrin, lactoferrin, Engrailed, Hoxa-5, Hoxb-4, or Hoxc-8. Human beta-3 integrin comprises PTDs that are hydrophobic signal sequence moieties. Engrailed-1, Engrailed-2, Hoxa-5, Hoxb-4 and Hoxc-8 are homeoproteins. Homeoproteins are helix turn helix proteins that contain a 60 amino acid DNA-binding domain, the homeodomain (HD). The PTD is believed to lie within the HD. When Engrailed-1 and Engrailed-2 are expressed in COS7 cells, they are first secreted and then reinternalized by other cells. Similar observations have been made for Hoxa-5, Hoxc-8 and Hoxb-4.

In some embodiments, the translocator is a herpes simplex virus type 1 (HSV-1) VP22 protein, which is a transcription factor that concentrates in the nucleus and binds chromatin. It has been shown that VP22 traffics across the membrane via non-classical endocytosis and can enter cells regardless of GAP junctions and physical contacts. If VP22 is expressed in a small population of cells in culture, it will reach 100% of the cells in that culture. Fusion proteins with VP22 and for example p53, GFP, thymidine kinase, β-galactosidase and others have been generated. It has been demonstrated that the fusion proteins are taken up by several kinds of cells including terminally differentiated cells suggesting that mitosis is not a requirement for efficient entry. In addition, VP22-GFP fusion showed that the protein can shuttle in and out of the cells and enter cells that were not exposed to VP22.

The HIV-1 trans-activator gene product (TAT) was one of the earliest cell-permeant proteins described. A receptor-mediated event is not required for TAT to pass into a neighboring cell. HIV-1, as well as other lentiviruses, encodes a potent Tat. The PTD of TAT is a small peptide comprising amino acids 47-57 or at least amino acids 49-57. Protein translational fusions with this 11 amino acid peptide can transit across the plasma membrane in vitro and in vivo. Proteins from 15 to 120 KDa have been tested and all enter human and murine cells efficiently. Schwartz, J J et al., Peptide-mediated cellular delivery, Curr Opin Mol Therapeutics 2000, 2:162-7. The disclosures of these references are incorporated in their entirety by reference herein. Furthermore, those proteins and peptides retain their biological properties and functions once inside the cells. In addition, the TAT-PTD is able to carry a variety of cargo molecules including nucleic acids (DNA and RNA), and therapeutic drugs. The capability of this sequence to internalize is dependent on the positive charges, and was not inhibited at 4° C. or in the presence of endocytosis inhibitors. The PTD sequence is able to mediate the transduction of its cargo in a concentration dependent and receptor-, transporter-, and endocytosis-independent manner to 100% of the target cells. Of special interest are the studies demonstrating that the PTD of TAT is able to deliver proteins in vivo to several tissues when injected into animals. A fusion protein of TAT-PTD and β-galactosidase was prepared and injected it into the peritoneum of mice. The presence of β-galactosidase activity in several tissues, including the brain, was demonstrated 4 hours after the intraperitoneal injection. Activity in the brain suggested that the fusion protein can also cross the blood-brain barrier. The studies have suggested that TAT-PTD fusion proteins are more efficiently transported inside cells and tissues when they are added exogenously in a denatured state. Their hypothesis is that they internalize easier than the folded protein and once inside the cell they are correctly refolded by chaperones and the target protein or peptide becomes fully active.

In some embodiments, the translocator comprises at least one PTD (PTD). Non-limiting examples of PTDs are shown on Table 1.

TABLE 1

| | | SEQ |
|---|---|---|
| | | ID |
| PTD | Sequence | NO. |
| Kaposi fibroblast growth factor | AAVALLPAVLLALLAP | 1 |
| membrane-translocating sequence | | |
| (kFGF MTS) | | |
| nuclear localization signal (NLS) | TPPKKKRKVEDP | 2 |
| Transportan | GWTLNSAGYLLGKIN | 3 |
| | LKALAALAKKIL | |
| herpes simplex virus type 1 protein | DAATATRGRSAASRP | 4 |
| 22 (VP22) | TERPRAPARSASRPR | |
| | RPVE | |
| human immunodeficiency virus | YGRKKRRQRRR | 5 |
| transactivator protein (TAT, 47-57) | | |

In some embodiments, PTDs of this invention are peptides derived from a homeoprotein. Homeoproteins are helix turn helix proteins that contain a 60 amino acid DNA-binding domain, the homeodomain (HD). PTDs may be derived from the HD. In some embodiments, PTDs are derived from the family of Drosophila homeoproteins. Drosophila homeoproteins are involved in developmental processes and are able to translocate across neuronal membranes. The third helix of the homeodomain of just 16 amino acids, known as penetratin, is able to translocate molecules into live cells. When added to several cell types in culture, 100% of the cells were able to uptake the peptide. Internalization occurs both at 37° C. and 4° C., and thus is neither receptor-mediated nor energy-dependent. Several penetrating peptides, the Penetratin family (Table 2) have been developed and used to internalize cargo molecules into the cytoplasm and nucleus of several cell types in vivo and in vitro. The results suggest that the entry of penetratin peptides relies on key tryptophan and, phenylalanine, and glutamine residues. In addition, the retroinverse and all D-amino acid forms are also translocated efficiently, and non α-helical structures are also internalized. See Prochiantz, A., Messenger proteins:homeoproteins, TAT and others, Curr Opin Cell Biol 2000, 12:400-6; and Schwartz, J J et al., Peptide-mediated cellular delivery, Curr Opin Mol Therapeutics 2000, 2:162-7. The disclosures of these references are incorporated in their entirety by reference herein.

In some embodiments, the translocator comprises at least one penetratin peptide. Non-limiting examples of penetratin peptides are shown on Table 2.

TABLE 2

| Sequence | SEQ ID NO. |
|---|---|
| RQIKIWFQNRRMKWKK | 6 |
| KKWKMRRNQFWIKIQR | 7 |
| RQIKIWFQNRRMKWKK | 8 |
| RQIKIWFPNRRMKWKK | 9 |
| RQPKIWFPNRRMPWKK | 10 |
| RQIKIWFQNMRRKWKK | 11 |
| RQIRIWFQNRRMRWRR | 12 |
| RRWRRWWRRWWRRWRR | 13 |
| RQIKIFFQNRRMKFKK | 14 |
| TERQIKIWFQNRRMK | 15 |
| KIWFQNRRMKWKKEN | 16 |

In some embodiments, a translocator comprises a synthetic protein transduction domain. Other synthetic PTD sequences that may be employed in accordance with the present invention may be found in WO 99/29721 and Ho, A. et al., Synthetic PTDs: enhanced transduction potential in vitro and in vivo, Cancer Res 2001, 61, 474-7. In addition, it has been demonstrated that a 9-mer of L-Arginine is 20 fold more efficient than the TAT-PTD at cellular uptake, and when a D-arginine oligomer was used the rate enhancement was >100 fold. See Wender, P A et al., The design, synthesis, and evaluation of molecules that enable or enhance cellular uptake: Peptoid molecular transporters, Proc. Natl. Acad. Sci. USA 2000, 97:13003-13008. These data suggested that the guanidinium groups of TAT-PTD play a greater role than charge or backbone structure in mediating cellular uptake. Thus, a peptoid analogue containing a six-methylene spacer between the guanidine head group and backbone was synthesized. This peptoid exhibited enhanced cellular uptake when compared to TAT-PTD and even to the D-Arg peptide.

In addition to the proteins and peptides discussed above, other peptide-mediated delivery systems have been described: MPG, SCWKn, (LARL)ₙ, HA2, RGD, AlkCWK₁₈, DiCWK₁₈, DipaLytic, K₁₆RGD, Plae and Kplae. See Schwartz, J J et al., Peptide-mediated cellular delivery, Curr Opin Mol Therapeutics 2000, 2:162-7. The disclosure of which is incorporated in its entirety by reference herein. In some embodiments, these proteins and peptides may be used as translocators in accordance with the present invention.

In some embodiments, a translocator comprises one or more of the sequence identified in Table 1 of Kabouridis et al., Biological applications of protein transduction technology, Trends in Biotechnology, Vol 21 No 11 November 2003, the disclosure of which is incorporated in its entirety herein by reference.

In some embodiments, a toxin of the present invention comprises a light chain. The light chain may be a light chain of a botulinum toxin, a butyricum toxin, a tetani toxin or biologically active variants of these toxins. In some embodiments, the light chain is a light chain of a botulinum toxin type A, B, C₁, D, E, F, G or biologically active variants of these serotypes. In some embodiments, a light chain of this invention is not cytotoxic—that is, its effects are reversible.

In some embodiments, the light chain of the present invention is about more than 75% homologous to the amino acid sequence of a wild type botulinum toxin serotype A, B, C1, D, E, F, or G. In some embodiments, the light chain of the present invention is about more than 85% homologous to the amino acid sequence of a wild type botulinum toxin serotype A, B, C1, D, E, F, or G. In some embodiments, the light chain of the present invention is about more than 95% homologous to the amino acid sequence of a wild type botulinum toxin serotype A, B, C1, D, E, F, or G. Percent homology can be determined by, for example, the Gap program (Wisconsin Sequence Analysis Package, Version 8 for Unix, Genetics Computer Group, University Research Park, Madison Wis.), which uses the algorithm of Smith and Waterman (Adv. Appl. Math., 1981, 2, 482-489, which is incorporated herein by reference in its entirety) using the default settings.

In some embodiments, a toxin of the present invention comprises a light chain and a heavy chain. The heavy chain may be a heavy chain of a botulinum toxin, a butyricum toxin, a tetani toxin. In some embodiments, the heavy chain is a heavy chain of a botulinum toxin type A, B, C₁, D, E, F or G. In some embodiments, the heavy chain of the present invention is about more than 75% homologous to the amino acid sequence of a wild botulinum toxin serotype A, B, C1, D, E, F, or G. In some embodiments, the heavy chain of the present invention is about more than 85% homologous to the amino acid sequence of a wild botulinum toxin serotype A, B, C1, D, E, F, or G. In some embodiments, the heavy chain of the present invention is about more than 95% homologous to the amino acid sequence of a wild botulinum toxin serotype A, B, C1, D, E, F, or G.

In some embodiments, the compound of the present invention is free of a carboxyl terminal of a heavy chain. In some embodiments, the compound of the present invention is free of a heavy chain.

Table 3 shows the light chain and heavy chain amino acid sequence of the wild type botulinum toxin that may be employed in accordance with the present invention.

TABLE 3

| Toxin | | | | |
|---|---|---|---|---|
| Accession | amino acid sequence of | SEQ | | SEQ |
| No. | LC | ID # | amino acid sequence of HC | ID # |
| BoNT/A | MPFVNKQFNYKDPVNGVDIAYI | 18 | ALDNDLCIKVNNWDLFFSPSEDNFTNDL | 19 |
| AF488749 | KIPNAGQMQPVKAFKIHNKIWV | | NKGEEITSDTNIEAAEENISLDLIQQYY | |
| | IPERDTFTNPEEGDLNFPPEAK | | LTFNFDNEPENISIENLSSDIIGQLELM | |
| | QVPVSYYDSTYLSTDNEKDNYL | | PNIERFPNGKKYELDKYTMFHYLRAQEF | |
| | KGVTKLFERIYSTDLGRMLLTS | | EHGKSRIALTNSVNEALLNPSRVYTFFS | |
| | IVRGIPFWGGSTIDTELKVIDT | | SDYVKKVNKATEAAMFLGWVEQLVYDFT | |
| | NCINVIQPDGSYRSEELNLVII | | DETSEVSTTDKIADITITIPYIGPALNI | |
| | GPSADIIQFECKSFGHEVLNLT | | GNMLYKDDFVGALTFSGAVILLEFIPEI | |
| | RNGYGSTQYIRFSPDFTFGFEE | | AIPVLGTFALVSYIANKVLTVQTTDNAL | |
| | SLEVDTNPLLGAGKFATDPAVT | | SKRNEKWDEVYKYIVTNWLAKVNTQIDL | |
| | LAHELIHAGHRLYGIAINPNRV | | IRKKMKEALENQAEATKAIINYQYNQYT | |
| | FKVNTNAYYEMSGLEVSFEELR | | EEEKNNINFNIDDLSSKLNESINKAMIN | |
| | TFGGHDAKFIDSLQENEFRLYY | | INKFLNQCSVSYLMNSMIPYGVKRLEDF | |
| | YNKFKDIASTLNKAKSIVGTTA | | DASLKDALLKYIYDNRGTLIGQVDRLKD | |
| | SLQYMKNVFKEKYLLSEDTSGK | | KVNNTLSTDIPFQLSKYVDNQRLLSTFT | |
| | FSVDKLKFDKLYKMLTEIYTED | | EYIKNIINTSTLNLRYESNHLIDLSRYA | |
| | NFVKFFKVLNRKTYLNFDKAVF | | SKINIGSKVNFDPIDKNQIQLFNLESSK | |
| | KINIVPKVNYTIYDGFNLRNT | | IEVILKNATVYNSMYENFSTSFWIRIPK | |
| | NLAANFNGQNTEINNMNFT | | YFNSISLNNEYTIINCMENNSGWKVSLN | |
| | KLKNFTGLFEFYKLLCVRG | | YGEIIWTLQDTQEIKQRVVFKYSQMINI | |
| | IITSK | | SDYINRWIFVTITNNRLNNSKIYINGRL | |
| | | | IDQKPISNLGNIHASNNIMFKLDGCRDT | |
| | | | HRYIWIKYFNLFDKELNEKEIKDLYDNQ | |
| | | | SNSGILKDFWGDYLQYDKPYYMLNLYDP | |
| | | | NKYVDVNNVGIRGYMYLKGPRGSVMTTN | |
| | | | IYLNSSLYRGTKFIIKKYASGNKDNIVR | |
| | | | NNDRVYINVVVKNKEYRLATNASQAGVE | |
| | | | KILSALEIPDVGNLSQVVVMKSKNDQGI | |
| | | | TNKCKMNLQDNNGNDIGFIGFHQFNNIA | |
| | | | KLVASNWYNRQIERSSRTLGCSWEFIPV | |
| | | | DDGWGERPL | |
| BoNT/B | PVTINNFNYNDPIDNDNII | 20 | YTIEEGFNISDKNMGKEYRGQNKAINKQ | 21 |
| I40631 | MMEPPFARGTGRYYKAFKI | | AYEEISKEHLAVYKIQMCKSVK\|VPGIC | |
| | TDRIWIIPERYTFGYKPED | | IDVDNENLFFIADKNSFSDDLSKNERVE | |
| | FNKSSGIFNRDVCEYYDPD | | YNTQNNYIGNDFPINELILDTDLISKIE | |
| | YLNTNDKKNIFFQTLIKLF | | LPSENTESLTDFNVDVPVYEKQPAIKKV | |
| | NRIKSKPLGEKLLEMIING | | FTDENTIFQYLYSQTFPLNIRDISLTSS | |
| | IPYLGDRRVPLEEFNTNIA | | FDDALLVSSKVYSFFSMDYIKTANKVVE | |
| | SVTVNKLISNPGEVERKKG | | AGLFAGWVKQIVDDFVIEANKSSTMDKI | |
| | IFANLIIFGPGPVLNENET | | ADISLIVPYIGLALNVGDETAKGNFESA | |
| | IDIGIQNHFASREGFGGIM | | FEIAGSSILLEFIPELLIPVVGVFLLES | |
| | QMKFCPEYVSVFNNVQENK | | YIDNKNKIIKTIDNALTKRVEKWIDMYG | |
| | GASIFNRRGYFSDPALILM | | LIVAQWLSTVNTQFYTIKEGMYKALNYQ | |
| | HELIHVLHGLYGIKVDDLP | | AQALEEIIKYKYNIYSEEEKSNININFN | |
| | IVPNEKKFFMQSTDTIQAE | | DINSKLNDGINQAMDNINDFINECSVSY | |
| | ELYTFGGQDPSIISPSTDK | | LMKKMIPLAVKKLLDFDNTLKKNLLNYI | |
| | SIYDKVLQNFRGIVDRLNK | | DENKLYLIGSVEDEKSKVDKYLKTIIPF | |
| | VLVCISDPNININIYKNKF | | DLSTYSNIEILIKIFNKYNSEILNNIIL | |
| | KDKYKFVEDSEGKYSIDVE | | NLRYRDNNLIDLSGYGAKVEVYDGVKLN | |
| | SFNKLYKSLMLGFTEINIA | | DKNQFKLTSSADSKIRVTQNQNIIFNSM | |
| | ENYKIKTRASYFSDSLPPV | | FLDFSVSFWIRIPKYRNDDIQNYIHNEY | |
| | KIKNLLDNEIYTIEEGFNI | | TIINCMKNNSGWKISIRGNRIIWTLIDI | |
| | SDKNMGKEYRGQNKAINKQ | | NGKTKSVFFEYNIREDISEYINRWFFVT | |
| | AYEEISKEHLAVYKIQMCK | | ITNNLDNAKIYINGTLESNMDIKDIGEV | |
| | SVK | | IVNGEITFKLDGDVDRTQFIWMKYFSIF | |
| | | | NTQLNQSNIKEIYKIQSYSEYLKDFWGN | |
| | | | PLMYNKEYYMFNAGNKNSYIKLVKDSSV | |
| | | | GEILIRSKYNQNSNYTNYRNLYIGEKFI | |
| | | | IRRESNSQSINDDIVRKEDYIHLDLVLH | |
| | | | HEEWRVYAYKYFKEQEEKLFLSIISDSN | |
| | | | EFYKTIEIKEYDEQPSYSCQLLFKKDEE | |
| | | | STDDIGLIGIHRFYESGVLRKKYKDYFC | |
| | | | ISKWYLKEVKRKPYKSNLGCNWQFIPKD | |
| | | | EGWTE | |
| BoNT/C1 | PITINNFNYSDPVDNKNIL | 22 | TLDCRELLVKNTDLPFIGDISDVKTDIF | 23 |
| P18640 | YLDTHLNTLANEPEKAFRI | | LRKDINEETEVIYYPDNVSVDQVILSKN | |
| | TGNIWVIPDRFSRNSNPNL | | TSEHGQLDLLYPSIDSESEILPGENQVF | |
| | NKPPRVTSPKSGYYDPNYL | | YDNRTQNVDYLNSYYYLESQKLSDNVED | |
| | STDSDKDTFLKEIIKLFKR | | FTFTRSIEEALDNSAKVYTYFPTLANKV | |
| | INSREIGEELIYRLSTDIP | | NAGVQGGLFLMWANDVVEDFTTNILRKD | |
| | FPGNNNTPINTFDFDVDFN | | TLDKISDVSAIIPYIGPALNISNSVRRG | |
| | SVDVKTRQGNNWVKTGSIN | | NFTEAFAVTGVTILLEAFPEFTIPALGA | |
| | PSVIITGPRENIIDPETST | | FVIYSKVQERNEIIKTIDNCLEQRIKRW | |
| | FKLTNNTFAAQEGFGALSI | | KDSYEWMMGTWLSRIITQFNNISYQMYD | |
| | ISISPRFMLTYSNATNDVG | | SLNYQAGAIKAKIDLEYKKYSGSDKENI | |
| | EGRFSKSEFCMDPILILMH | | KSQVENLKNSLDVKISEAMNNINKFIRE | |
| | ELNHAMHNLYGIAIPNDQT | | CSVTYLFKNMLPKVIDELNEFDRNTKAK | |
| | ISSVTSNIFYSQYNVKLEY | | LINLIDSHNIILVGEVDKLKAKVNNSFQ | |
| | AEIYAFGGPTIDLIPKSAR | | NTIPFNIFSYTNNSLLKDIINEYFNNIN | |
| | KYFEEKALDYYRSIAKRLN | | DSKILSLQNRKNTLVDTSGYNAEVSEEG | |
| | SITTANPSSFNKYIGEYKQ | | DVQLNPIFPFDFKLGSSGEDRGKVIVTQ | |
| | KLIRKYRFVVESSGEVTVN | | NENIVYNSMYESFSISFWIRINKWVSNL | |
| | RNKFVELYNELTQIFTEFN | | PGYTIIDSVKNNSGWSIGIISNFLVFTL | |
| | YAKIYNVQNRKIYLSNVYT | | KQNEDSEQSINFSYDISNNAPGYNKWFF | |
| | PVTANILDDNVYDIQNGFN | | VTVTNNMMGNMKIYINGKLIDTIKVKEL | |
| | IPKSNLNVLFMGQNLSRNP | | TGINFSKTITFEINKIPDTGLITSDSDN | |
| | ALRKVNPENMLYLFTKFCH | | INMWIRDFYIFAKELDGKDINILFNSLQ | |
| | KAIDGRSLYNK | | YTNVVKDYWGNDLRYNKEYYMVNIDYLN | |
| | | | RYMYANSRQIVFNTRRNNNDFNEGYKII | |
| | | | IKRIRGNTNDTRVRGGDILYFDMTINNK | |
| | | | AYNLFMKNETMYADNHSTEDIYAIGLRE | |
| | | | QTKDINDNIIFQIQPMNNTYYYASQIFK | |
| | | | SNFNGENISGICSIGTYRFRLGGDWYRH | |
| | | | NYLVPTVKQGNYASLLESTSTHWGFVPV | |
| | | | SE | |
| BoNT/D | MTWPVKDFNYSDPVNDNDI | 24 | NSRDDSTCIKVKNNRLPYVADKDSISQE | 25 |
| P19321 | LYLRIPQNKLITTPVKAFM | | IFENKIITDETNVQNYSDKFSLDESILD | |
| | ITQNIWVIPERFSSDTNPS | | GQVPINPEIVDPLLPNVNMEPLNLPGEE | |
| | LSKPPRPTSKYQSYYDPSY | | IVFYDDITKYVDYLNSYYYLESQKLSNN | |
| | LSTDEQKDTFLKGIIKLFK | | VENITLTTSVEEALGYSNKIYTFLPSLA | |
| | RINERDIGKKLINYLVVGS | | EKVNKGVQAGLFLNWANEVVEDFTTNIM | |
| | PFMGDSSTPEDTFDFTRHT | | KKDTLDKISDVSVIIPYIGPALNIGNSA | |
| | TNIAVEKFENGSWKVTNII | | LRGNFNQAFATAGVAFLLEGFPEFTIPA | |
| | TPSVLIFGPLPNILDYTAS | | LGVFTFYSSIQEREKIIKTIENCLEQRV | |
| | LTLQGQQSNPSFEGFGTLS | | KRWKDSYQWMVSNWLSRITTQFNHINYQ | |
| | ILKVAPEFLLTFSDVTSNQ | | MYDSLSYQADAIKAKIDLEYKKYSGSDK | |
| | SSAVLGKSIFCMDPVIALM | | ENIKSQVENLKNSLDVKISEAMNNINKF | |
| | HELTHSLHQLYGINIPSDK | | IRECSVTYLFKNMLPKVIDELNKFDLRT | |
| | RIRPQVSEGFFSQDGPNVQ | | KTELINLIDSHNIILVGEVDRLKAKVNE | |
| | FEELYTFGGLDVEIIPQIE | | SFENTMPFNIFSYTNNSLLKDIINEYFN | |
| | RSQLREKALGHYKDIAKRL | | SINDSKILSLQNKKNALVDTSGYNAEVR | |
| | NNINKTIPSSWISNIDKYK | | VGDNVQLNTIYTNDFKLSSSGDKIIVNL | |
| | KIFSEKYNFDKDNTGNFVV | | NNNILYSAIYENSSVSFWIKISKDLTNS | |
| | NIDKFNSLYSDLTNVMSEV | | HNEYTIINSIEQNSGWKLCIRNGNIEWI | |
| | VYSSQYNVKNRTHYFSRHY | | LQDVNRKYKSLTFDYSESLSHTGYTNKW | |
| | LPVFANILDDNIYTIRDGF | | FFVTITNNIMGYMKLYINGELKQSQKIE | |
| | NLTNKGFNIENSGQNIERN | | DLDEVKLDKTIVFGIDENIDENQMLWIR | |
| | PALQKLSSESVVDLFTKVC | | DFNIFSKELSNEDINIVYEGQILRNVIK | |
| | LRLTK | | DYWGNPLKFDTEYYIINDNYIDRYIAPE | |
| | | | SNVLVLVQYPDRSKLYTGNPITIKSVSD | |
| | | | KNPYSRILNGDNIILHMLYNSRKYMIIR | |
| | | | DTDTIYATQGGECSQNCVYALKLQSNLG | |
| | | | NYGIGIFSIKNIVSKNKYCSQIFSSFRE | |
| | | | NTMLLADIYKPWRFSFKNAYTPVAVTNY | |
| | | | ETKLLSTSSFWKFISRDPGWVE | |
| BoNT/E | PKINSFNYNDPVNDRTILY | 26 | SICIEINNGELFFVASENSYNDDNINTP | 27 |
| P30995 | IKPGGCQEFYKSFNIMKNI | | KEIDDTVTSNNNYENDLDQVILNFNSES | |
| | WIIPERNVIGTTPQDFHPP | | APGLSDEKLNLTIQNDAYIPKYDSNGTS | |
| | TSLKNGDSSYYDPNYLQSD | | DIEQHDVNELNVFFYLDAQKVPEGENNV | |
| | EEKDRFLKIVTKIFNRINN | | NLTSSIDTALLEQPKIYTFFSSEFINNV | |
| | NLSGGILLEELSKANPYLG | | NKPVQAALFVSWIQQVLVDFTTEANQKS | |
| | NDNTPDNQFHIGDASAVEI | | TVDKIADISIVVPYIGLALNIGNEAQKG | |
| | KFSNGSQDILLPNVIIMGA | | NFKDALELLGAGILLEFEPELLIPTILV | |
| | EPDLFETNSSNISLRNNYM | | FTIKSFLGSSDNKNKVIKAINNALKERD | |
| | PSNHGFGSIAIVTFSPEYS | | EKWKEVYSFIVSNWMTKINTQFNKRKEQ | |
| | FRFNDNSMNEFIQDPALTL | | MYQALQNQVNAIKTIIESKYNSYTLEEK | |
| | MHELIHSLHGLYGAKGITT | | NELTNKYDIKQIENELNQKVSIAMNNID | |
| | KYTITQKQNPLITNIRGTN | | RFLTESSISYLMKLINEVKINKLREYDE | |
| | IEEFLTFGGTDLNIITSAQ | | NVKTYLLNYIIQHGSILGESQQELNSMV | |
| | SNDIYTNLLADYKKIASKL | | TDTLNNSIPFKLSSYTDDKILISYFNKF | |
| | SKVQVSNPLLNPYKDVFEA | | FKRIKSSSVLNMRYKNDKYVDTSGYDSN | |
| | KYGLDKDASGIYSVNINKF | | ININGDVYKYPTNKNQFGIYNDKLSEVN | |
| | NDIFKKLYSFTEFDLATKF | | ISQNDYIIYDNKYKNFSISFWVRIPNYD | |
| | QVKCRQTYIGQYKYFKLSN | | NKIVNVNNEYTIINCMRDNNSGWKVSLN | |
| | LLNDSIYNISEGYNINNLK | | HNEIIWTLQDNAGINQKLAFNYGNANGI | |
| | VNFRGQNANLNPRIITPIT | | SDYINKWIFVTITNDRLGDSKLYINGNL | |
| | GRGLVKKIIRFCKNIVSVK | | IDQKSILNLGNIHVSDNILFKIVNCSYT | |
| | GIRK | | RYIGIRYFNIFDKELDETEIQTLYSNEP | |
| | | | NTNILKDFWGNYLLYDKEYYLLNVLKPN | |
| | | | NFIDRRKDSTLSINNIRSTILLANRLYS | |
| | | | GIKVKIQRVNNSSTNDNLVRKNDQVYIN | |
| | | | FVASKTHLFPLYADTATTNKEKTIKISS | |
| | | | SGNRFNQVVVMNSVGNNCTMNFKNNNGN | |
| | | | NIGLLGFKADTVVASTWYYTHMRDHTNS | |
| | | | NGCFWNFISEEHGWQEK | |
| BoNT/F | MPVAINSFNYNDPVNDDTI | 28 | GTKAPPRLCTRVNNSELFFVASESSYNE | 29 |
| P30996 | LYMQIPYEEKSKKYYKAFE | | NDINTPKETDDTTNLNNNYRNNLDEVIL | |
| | IMRNVWIIPERNTIGTNPS | | DYNSQTIPQISNRTLNTLVQDNSYVPRY | |
| | DFDPPASLKNGSSAYYDPN | | DSNGTSEIEEYDVVDFNVFFYLHAQKVP | |
| | YLTTDAEKDRYLKTTIKLF | | EGETNISLTSSIDTALLEESKDIFFSSE | |
| | KRINSNPAGKVLLQEISYA | | FIDTINKPVNAALFIDWISKVIRDFTTE | |
| | KPYLGNDHTPIDEFSPVTR | | ATQKSTVDKIADISLIVPYVGLALNIII | |
| | TTSVNIKLSTNVESSMLLN | | EAEKGNFEEAFELLGVGILLEFVPELTI | |
| | LLVLGAGPDIFESCCYPVR | | PVILVFTIKSYIDSYENKNKAIKAINNS | |
| | KLIDPDVVYDPSNYGFGSI | | LIEREAKWKEIYSWIVSNWLTRINTQFN | |
| | NIVTFSPEYEYTFNDISGG | | KRKEQMYQALQNQVDAIKTAIEYKYNNY | |
| | HNSSTESFIADPAISLAHE | | TSDEKNRLESEYNINNIEEELNKKVSLA | |
| | LIHALHGLYGARGVTYEET | | MKNIERFMTESSISYLMKLINEAKVGKL | |
| | IEVKQAPLMIAEKPIRLEE | | KKYDNHVKSDLLNYILDHRSILGEQTNE | |
| | FLTFGGQDLNIITSAMKEK | | LSDLVTSTLNSSIFFELSSYTNDKILII | |
| | IYNNLLANYEKIATRLSEV | | YFNRLYKKIKDSSILDMRYENNKFIDIS | |
| | NSAPPEYDINEYKDYFQWK | | GYGSNISINGNVYIYSTNRNQFGIYNSR | |
| | YGLDKNADGSYTVNENKFN | | LSEVNIAQNNDIIYNSRYQNFSTSFWVR | |
| | EIYKKLYSFTESDLANKFK | | IPKHYKPMNHNREYTIINCMGNNNSGWK | |
| | VKCRNTYFIKYEFLKVPNL | | ISLRTVRDCEIIWTLQDTSGNKENLIFR | |
| | LDDDIYTVSEGFNIGNLAV | | YEELNRISNYINKWIFVTITNNRLGNSR | |
| | NNRGQSIKLNPKIIDSIPD | | IYINGNLIVEKSISNLGDIHVSDNILFK | |
| | KGLVEKIVKFCKSVIPRK | | IVGCDDETYVGIRYFKVFNTELDKTEIE | |
| | | | TLYSNEPDPSILKNYWGNYLLYNKKYYL | |
| | | | FNLLRKDKYITLNSGILNINQQRGVTEG | |
| | | | SVFLNYKLYEGVEVIIRKNGPIDISNTD | |
| | | | NFVRKNDLAYINVVDRGVEYRLYADTKS | |
| | | | EKEKIIRTSNLNDSLGQIIVNDSIGNNC | |
| | | | TMNFQNNNGSNIGLLGFHSNNLVASSWY | |
| | | | YNNIRRNTSSNGCFWSSISKENGWKE | |
| BoNT/G | PVNIKXFNYNDPINNDDII | 30 | NTGKSEQCIIVNNEDLFFIANKDSFSKD | 31 |
| Q60393 | MMEPFNDPGPGTYYKAFRI | | LAKAETIAYNTQNNTIENNFSIDQLILD | |
| | IDRIWIVPERFTYGFQPDQ | | NDLSSGIDLPNENTEPFTNFDDIDTPVY | |
| | FNASTGVFSKDVYEYYDPT | | IKQSALKKIFVDGDSLFEYLHAQTFPSN | |
| | YLKTDAEKDKFLKTMIKLF | | IENLQLTNSLNDALRNNNKVYTFFSTNL | |
| | NRINSKPSGQRLLDMIVDA | | VEKANTVVGASLFVNWVKGVIDDFTSES | |
| | IPYLGNASTPPDKFAANVA | | TQKSTIDKVSDVSIIIPYIGPALNVGNE | |
| | NVSINKKIIQPGAEDQIKG | | TAKENFKNAFEIGGAAILMEFIPELIVP | |
| | LMTNLIIFGPGPVLSDNFT | | IVGFFTLESYVGNKGHIIMTISNALKKR | |
| | DSMIMNGHSPISEGFGARM | | DQKWTDMYGLIVSQWLSTVNTQFYTIKE | |
| | MIRFCPSCLNVFNNVQENK | | RMYNALNNQSQAIEKIIEDQYNRYSEED | |
| | DTSIFSRRAYFADPALTLM | | KMNINIDFNDIDFKLNQSINLAINNIDD | |
| | HELIHVLHGLYGIKISNLP | | FINQCSISYLMNRMIPLAVKKLKDFDDN | |
| | ITPNTKEFFNQHSDPVQAE | | LKRDLLEYIDTNELYLLDEVNILKSKVN | |
| | ELYTFGGHDPSVISPSTDM | | RHLKDSIPFDLSLYTKDTILIQVFNNYI | |
| | NIYNKALQNFQDIANRLNI | | SNISSNAILSLSYRGGRLIDSSGYGATM | |
| | VSSAQGSGIDISLYKQIYK | | NVGSDVIFNDIGNGQFKLNNSENSNITA | |
| | NKYDFVEDPNGKYSVDKDK | | HQSKFVVYDSMFDNFSINFWVRTPKYNN | |
| | FDKLYKALMFGFTETNLAG | | NDIQTYLQNEYTIISCIKNDSGWKVSIK | |
| | EYGIKTRYSYFSEYLPPIK | | GNRIIWTLIDVNAKSKSIFFEYSIKDNI | |
| | TEKLLDNTIYTQNEGFNIA | | SDYINKWFSITITNDRLGNANIYINGSL | |
| | SKNLKTEFNGQNKAVNKEA | | KKSEKILNLDRINSSNDIDFKLINCTDT | |
| | YEEISLEHLVIYRIAMCKP | | TKFVWIKDFNIFGRELNATEVSSLYWIQ | |
| | VMYK | | SSTNTLKDFWGNPLRYDTQYYLFNQGMQ | |
| | | | NIYIKYFSKASMGETAPRTNFNNAAINY | |
| | | | QNLYLGLRFIIKKASNSRNINNDNIVRE | |
| | | | GDYIYLNIDNISDESYRVYVLVNSKEIQ | |
| | | | TQLFLAPINDDPTFYDVLQIKKYYEKTT | |
| | | | YNCQILCEKDTKTFGLFGIGKFVKDYGY | |
| | | | VWDTYDNYFCISQWYLRRISENINKLRL | |
| | | | GCNWQFIPVDEGWTE | |

In some embodiments, a toxin of the present invention may comprise any combination of light chain and heavy chain. In some embodiment, a toxin of the present invention may comprise a light chain and a heavy chain of the same serotype. For example, a toxin of the present invention may comprise a botulinum toxin light chain serotype A and a botulinum toxin heavy chain serotype A. In some embodiments, a toxin may comprise a light chain and a heavy chain of different serotypes. For example, toxin of the present invention may comprise a light chain serotype A and a heavy chain serotype E.

One or more translocators may be linked to any amino acid residue of a toxin. For example, a translocator may be linked to the N-terminal residue, the C-terminal residue or any residue along any non critical region of a toxin, e.g., a light chain, as long as the toxicity of the toxin is not substantially reduced. The non-critical regions of incorporation may be determined experimentally by assessing the resulting toxicity of the modified toxin using standard toxicity assays such as that described by Zhou, L., et al., Biochemistry (1995) 34:15175-15181.

In some embodiments, a toxin of the present invention comprises a botulinum toxin type A linked to a human immunodeficiency virus transactivator protein peptide (SEQ ID NO: 5). In some embodiments, the light chain of the botulinum toxin is linked to the human immunodeficiency virus transactivator protein peptide (SEQ ID NO: 5). In some embodiments, the heavy chain of the botulinum toxin is linked to the human immunodeficiency virus transactivator protein peptide (SEQ ID NO: 5). In some embodiments, this toxin is further linked to a targeting moiety. For example, the targeting moiety may be linked to the toxin or the human immunodeficiency virus transactivator protein peptide (SEQ ID NO: 5).

In some embodiments, a toxin of the present invention comprises a light chain of botulinum toxin type A linked to a human immunodeficiency virus transactivator protein peptide (SEQ ID NO: 5). In some embodiments, the N-terminus of the light chain of the botulinum toxin is linked to the human immunodeficiency virus transactivator protein peptide (SEQ ID NO: 5). In some embodiments, the C-terminus of the light chain of the botulinum toxin is linked to the human immunodeficiency virus transactivator protein peptide (SEQ ID NO: 5). In some embodiments, this toxin is further linked to a targeting moiety. For example, the targeting moiety may be linked to the toxin or the human immunodeficiency virus transactivator protein peptide (SEQ ID NO: 5).

In some embodiments, one toxin is linked to one translocator. For example, a compound of the present invention may comprise a translocator linked to a C-terminal or N-terminal of a toxin, e.g., a light chain. In some embodiments, more than one toxin is linked to a translocator. For example, a compound of the present invention comprises a toxin linked to a translocator peptide at the N and C terminal of the translocator peptide. In some embodiments, a toxin is linked to more than one translocator. For example, a compound of the present invention may comprise light chain linked to a first translocator at the N-terminal of the light chain, and a second translocator linked to the C-terminal of the same light chain.

In some embodiments, the compounds of the present invention comprise a toxin linked to a translocator and a targeting moiety. As defined above, a targeting moiety is a chemical compound or a peptide that is able to bind to a specific cell surface receptor. In some embodiments, the targeting moiety directs the compound to the appropriate cells, and the translocator facilitates the transport of the compound into those particular cells. A non-limiting example of a targeting moiety include substance-P for directing the compounds to sensory nerve terminals. In some embodiments, the compound of the present invention comprising a substance-P targeting moiety may be administered to treat pain. In some embodiments, the compound of the present invention comprising a CCK targeting moiety may be administered to treat pancreatitis. In some embodiments, the compound of the present invention comprising an eosinophil targeting moiety may be administered to treat allergies. In some embodiments, the compound of the present invention comprising a sweat gland targeting moiety may be administered to treat hyperhidrosis.

In some embodiments, a compound comprising a translocator translocate about more than 10% more of the toxin into a cell as compared to an identical compound that does not comprise a translocator. In some embodiments, a compound comprising a translocator translocates about more than 25% more of the toxin into a cell as compared to an identical compound that does not comprise a translocator. In some embodiments, a compound comprising a translocator translocates about more than 50% more of the toxin into a cell as compared to an identical compound that does not comprise a translocator. In some embodiments, a compound comprising a translocator translocates about more than 100% more of the toxin into a cell as compared to an identical compound that does not comprise a translocator.

In some embodiments, a compound of the present invention comprises a light chain of botulinum toxin type A and TAT (SEQ ID NO: 5), wherein the TAT is linked at the N or C terminal of the light chain. In some embodiments, a compound of the present invention comprises a light chain of botulinum toxin type A, a TAT (SEQ ID NO: 5), and a targeting moiety; wherein the TAT and targeting moiety are linked at the C and N terminal of the light chain, respectively.

In some embodiments, the compounds of the present invention comprise one or more protease cleavage domain. In this aspect, the protease cleavage site must be engineered so that it does not substantially affect the toxicity of the compound that it is a part of, but when cleaved, will result in a substantially non-toxic compound fragment. Accordingly, the term “does not substantially affect the toxicity” means that a compound containing the protease cleavage domain is at least 10%, preferably 25%, more preferably, 50%, more preferably 75% and even more preferably at least 90% as toxic as a compound not containing the protease cleavage site. Compounds comprising a protease cleavage domain that have toxic activity greater than compounds without a protease cleavage domain are also included in this invention. The non-critical regions of incorporation may be determined experimentally by assessing the resulting toxicity of the modified toxin using standard toxicity assays such as that described by Zhou, L., et al., Biochemistry (1995) 34:15175-15181. See also U.S. patent application Ser. No. 726949, filed Nov. 29, 2000, and published on Sep. 26, 2002 as U.S. application 2002 0137886. The disclosure of this application is incorporated in its entirety herein by reference.

In order to be operative for purposes of this invention, when the protease cleavage domain is cleaved, the toxic activity of the compound is substantially diminished. In this context, “substantially diminished” means that the toxin retains less than 50% of the original toxicity, or more preferably less than 25% of the toxicity, even more preferably 10% of the activity. In some embodiments, when the protease cleavage domain is cleaved, the toxic activity of the compound is less than 1% of the activity, as compared to the same compound that is not cleaved.

In some embodiments, the protease cleave domain is located between the toxin and the translocator. Accordingly, a cleavage of the compound results in a separation of the toxin from the translocator. As such, the toxin would not be able to translocate into a cell, resulting in a partial or complete loss of toxicity of the compound.

In some embodiments, a compound comprising a clostridial toxin linked to a translocator may have more than one cleavage domain. For example, a compound comprising a clostridial toxin with a linear N to C-sequence of heavy chain—light chain—translocator may have a cleavage domain be engineered between the heavy chain and light chain and an additional cleavage site be engineered between the light chain and the translocator.

For the design of a compound which can be inactivated by blood, protease sites which are recognized by proteases relatively uniquely found in the bloodstream are desirable. Among these proteases are those set forth below in Table 4, which also describes their recognition sites.

TABLE 4
Proteases Present in Blood

| Blood protease | Substrate Specificity |
|---|---|
| Thrombin | P4-P3-P-R/K*P1′-P2′ P3/P4 hydrophobic; |
| | P1′/P2′ non-acidic |
| | P2-R/K*P1′ P2 or P1′ are G |
| Coagulation Factor Xa | I-E/D-G-R* |
| Coagulation Factor | R* |
| XIa | |
| Coagulation Factor | R* |
| XIIa | |
| Coagulation Factor | R* |
| IXa | |
| Coagulation Factor | R/K* |
| VIIa | |
| Kallikrein | R/K* |
| Protein C | R* |
| MBP-associated | R* |
| serine protease | |
| Oxytocinase | N-terminal C* |
| Lysine | C-terminal R/K* |
| carboxypeptidase | |
| ADAM-TS13 | Substrate is VWF at the Y1605-M1606 |
| | bond. |
| | Substrate describe is D1596-R1668 of the |
| | VWF |

*indicates the peptide bond this protease will cleave.

Coagulation factors XIa, XIIa, IXa and VIIa as well as kallikrein, protein C, MBP-associated serine protease, oxytocinase and lysine carboxypeptidase have relatively nonspecific target sites, while coagulation factors Xa, ADAM-TS13, and thrombin provide the opportunity for more specificity. In designing a thrombin, VWF, or coagulation factor Xa site into a compound of the present invention, the location of the inserted site is, as described above, such that the presence of the site will not interfere with activity of the toxin, but cleavage at the site will destroy or vastly inhibit the activity of the toxin.

In some embodiments, a protease cleavage domain may be located within the targeting moiety or the translocator, but away from the functional domains within these regions. Insertion sites in the targeting moiety should be away from receptor binding grooves and in all cases the sites should be selected so as to be on the surface of the protein so that blood proteases can freely access them.

Thus, for the inactivating cleavage, the protease should be one present in high levels in blood. A suitable protease in this regard is thrombin, which occurs in blood in levels sufficient to deactivate the modified form of the toxins herein. By “effective” level of the protease is meant a concentration which is able to inactivate at least 50%, preferably 75%, more preferably 90% or greater of the toxin which enters the bloodstream at clinically suitable levels of dosage.

In general, the dosage levels for the present compounds are on the order of nanogram levels of concentration and thus are not expected to require higher concentrations of protease.

Although blood proteases are presently discussed, protease sites for non-blood proteases may be employed in accordance with this invention.

In some embodiments, the toxin and other components, e.g., the translocator and/or the targeting moiety, are linked by a covalent bond. For example, a compound may comprise a light chain having a direct covalent bond with a translocator. In some embodiments, chemical linkers (hereinafter “Linker Y” or “Y”) may be used to link together two or more components of the present compound. For example, a Linker Y may be used to link a light chain to a translocator.

Linker Y may be selected from the group consisting of 2-iminothiolane, N-succinimidyl-3-(2-pyridyldithio) propionate (SPDP), 4-succinimidyloxy carbonyl-alpha-(2-pyridyldithio)toluene (SMPT), m-maleimido benzoyl-N-hydroxysuccinimide ester (MBS), N-succinimidyl(4-iodoacetyl) aminobenzoate (SIAB), succinimidyl 4-(p-maleimidophenyl) butyrate (SMPB), 1-ethyl-3-(3-dimethylaminopropyl) carbodiimide (EDC), bis-diazobenzidine and glutaraldehyde.

In some embodiments, Linker Y may be attached to an amino group, a carboxylic group, a sulfhydryl group or a hydroxyl group of an amino acid group of a component. For example, a Linker Y may be linked to a carboxyl acid group of amino acid of a translocator.

In some embodiments, spacers may be used to physically further separate components of the present invention. For example, a compound of the present invention may comprise a light chain linked to a translocator through a spacer. In some embodiments, a spacer functions to create a distance between the components to minimize or eliminate steric hindrances to the components. In some embodiments, the minimization or elimination of steric hindrances allows the respective components to function more effectively.

In some embodiments, a spacer comprises a proline, serine, threonine and/or cysteine-rich amino acid sequence similar or identical to a human immunoglobulin hinge region. In some embodiments, the spacer comprises the amino acid sequence of an immunoglobulin g1 hinge region. Such a sequence has the sequence:

(SEQ ID NO: 32)

Glu-Pro-Lys-Ser-Cys-Asp-Lys-Thr-His-Thr-Cys-Pro-
Pro-Cys-Pro.

Spacers may also comprise hydrocarbon moieties. For example, such hydrocarbon moieties are represented by the chemical formulas:

HOOC—(CH₂)ₙ—COOH, where n=1-12 or,
HO—(CH₂)ₙ-COOH, where n>10

In some embodiments, a Linker Y may be used to link a light chain to a translocator. In another embodiment, a Linker Y may be employed to link an L to a spacer; in turn, that spacer may then be linked to a translocator by another Linker Y, forming a compound comprising the structure:
L-Y-spacer-Y-translocator.

Linker Y may be selected from the group consisting of 2-iminothiolane, N-succinimidyl-3-(2-pyridyldithio) propionate (SPDP), 4-succinimidyloxy carbonyl-alpha-(2-pyridyldithio)toluene (SMPT), m-maleimido benzoyl-N-hydroxysuccinimide ester (MBS), N-succinimidyl(4-iodoacetyl) aminobenzoate (SIAB), succinimidyl 4-(p-maleimidophenyl) butyrate (SMPB), 1-ethyl-3-(3-dimethylaminopropyl) carbodiimide (EDC), bis-diazobenzidine and glutaraldehyde.

In some embodiments, Linker Y may be attached to an amino group, a carboxylic group, a sulfhydryl group or a hydroxyl group of an amino acid group of a component. For example, a Linker Y may be linked to a carboxyl acid group of amino acid of a translocator.

Although the described chemistry may be used to couple the components of the described invention, any other coupling chemistry known to those skilled in the art capable of chemically attaching a targeting component to another component of a compound of the invention is covered by the scope of this invention.

Compounds of the present invention have potential utility in human medicine. For example, the compounds of the present invention may be administered for the treatment of biological disorders. The biological disorders that may be treated in accordance with the present invention include neuromuscular disorders, autonomic disorders and pain. In some embodiments, the method of treating a neuromuscular disorder comprises the locally administering a compound of the present invention to a group of muscles. In some embodiments, the method of treating an autonomic disorder comprises locally administering a compound of the present invention to a gland. In some embodiments, the method of treating pain comprises locally administering a compound of the present invention to the site of pain. In some embodiments, the method of treating pain comprises administering a compound of the present invention to a spinal cord. In some embodiments, the method of treating asthma or allergies comprises administering an aerosolized compound of the present invention to the target tissue or cell, e.g, respiratory tissues or mast cells.

The dose of the compound to be administered depends on many factors. For example, the better each one of the components is able to perform its respective function, the lower the dose of the compound is required to obtain a desired therapeutic effect. One of ordinary skill will be able to readily determine the specific dose for each specific compound. For compounds employing a natural, mutated or recombinant botulinum toxin A comprising the therapeutic, translocation and targeting component, an effective dose of an compound to be administered may be about 1 U to about 500 U of the botulinum toxin serotype A, or its equivalent. A dose of a non-botulinum toxin type A is an equivalent to a dose of botulinum toxin type A if they both have about the same degree of prevention or treatment when administered to a mammal (although their duration may differ). The degree of prevention or treatment may be measured by an evaluation of the improved patient function criteria set forth below.

Furthermore, the amount of the compounds administered can vary widely according to the particular disorder being treated, its severity and other various patient variables including size, weight, age, and responsiveness to therapy. Such determinations are routine to one of ordinary skill in the art (see for example, Harrison's Principles of Internal Medicine (1998), edited by Anthony Fauci et al., 14th edition, published by McGraw Hill).

Other routes of administration include, without limitation, transdermal, peritoneal, subcutaneous, intramuscular, intravenous, intrarectal and/or via inhalation (e.g., aerosolized compounds).

In some embodiments, recombinant techniques are used to produce at least one of the components of the compounds. See, for example International Patent Application Publication WO 95/32738, the disclosure of which is incorporated in its entirety herein by reference. The technique includes steps of obtaining genetic materials from DNA cloned from natural sources, or synthetic oligonucleotide sequences, which have codes for one of the components, for example the toxins, translocators and/or targeting moieties. The genetic constructs are incorporated into host cells for amplification by first fusing the genetic constructs with a cloning vector, such as a phage, plasmid, phagemid or other gene expression vector. The recombinant cloning vectors are transformed into a mammalian, insect cells, yeast or bacterial hosts. The preferred host is E. coli. Following expression of recombinant genes in host cells, resultant proteins can be isolated using conventional techniques. The protein expressed may comprise a toxin and a translocator fused together. For example, the protein expressed may include a light chain of botulinum toxin type A fused to a TAT. In some embodiments, the expressed proteins be separately expressed and are then chemically joined, for example, through linker Y.

The compounds of the invention may also be admixed, encapsulated, conjugated or otherwise associated with other molecules or mixtures of compounds as, for example, liposomes, formulations (oral, rectal, topical, etc.) for assisting in uptake, distribution and/or absorption.

Pharmaceutical compounds and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compounds of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Compounds of the invention may be encapsulated within liposomes or may form complexes thereto, in particular to cationic liposomes. Alternatively, compounds may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in United States patent application Ser. No. 09/315,298 filed on May 20, 1999 which is incorporated herein by reference in its entirety.

Compounds and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which compounds of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Compounds of the invention may be delivered orally, in granular form including sprayed dried particles, or complexed to form micro or nanoparticles. Compound complexing agents include

poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

Compounds and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compounds of the present invention include, but are not limited to, solutions, emulsions, and liposome-containing formulations. These compounds may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compounds of the present invention may be formulated into any of many possible dosage forms such as, but not limited to, tablets, capsules, gel capsules, liquid syrups, soft gels, suppositories, and enemas. The compounds of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compounds may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compounds and formulations is generally known to those skilled in the pharmaceutical and formulation arts and may be applied to the formulation of the compounds of the present invention.

The following non-limiting examples provide those of ordinary skill in the art with exemplary suitable methods for practicing the present invention, and are not intended to limit the scope of the invention.

### EXAMPLE 1

### Subcloninq the BoNT/A-L Chain Gene

This example describes an exemplary method to clone the polynucleotide sequence encoding the BoNT/A-L chain. The DNA sequence encoding the BoNT/A-L chain may be amplified by a PCR protocol that employs synthetic oligonucleotides having the sequences, 5′-AAAGGCCTTTTGTTAAT AAACAA-3′ (SEQ ID NO: 33) and 5′-GGMTTCTTACTTATTGTATCCTTTA-3′ (SEQ ID NO: 34). Use of these primers allows the introduction of Stu I and EcoR I restriction sites into the 5′ and 3′ ends of the BoNT/A-L chain gene fragment, respectively. These restriction sites may be subsequently used to facilitate unidirectional subcloning of the amplification products. Additionally, these primers introduce a stop codon at the C-terminus of the L chain coding sequence. Chromosomal DNA from C. botulinum (strain 63 A) may serve as a template in the amplification reaction.

The PCR amplification is performed in a 0.1 mL volume containing 10 mM Tris-HCl (pH 8.3), 50 mM KCl, 1.5 mM MgCl2, 0.2 mM of each deoxynucleotide triphosphate (dNTP), 50 pmol of each primer, 200 ng of genomic DNA and 2.5 units of Taq polymerase (Promega). The reaction mixture is subjected to 35 cycles of denaturation (1 minute at 94° C.), annealing (2 minutes at 37° C.) and polymerization (2 minutes at 72° C.). Finally, the reaction is extended for an additional 5 minutes at 72° C.

The PCR amplification product may be digested with Stu I and EcoR I, purified by agarose gel electrophoresis, and ligated into Sma I and EcoR I digested pBluescript II SK* to yield the plasmid, pSAL. Bacterial transformants harboring this plasmid may be isolated by standard procedures. The identity of the cloned L chain polynucleotide is confirmed by double stranded plasmid sequencing using SEQUENASE (United States Biochemicals) according to the manufacturer's instructions. Synthetic oligonucleotide sequencing primers are prepared as necessary to achieve overlapping sequencing runs. The cloned sequence is found to be identical to the sequence disclosed by Binz, et al., in J. Biol. Chem. 265, 9153 (1990), and Thompson et al., in Eur. J. Biochem. 189, 73 (1990). Site-directed mutants designed to compromise the enzymatic activity of the BoNT/A-L chain may also be created.

### EXAMPLE 2

### Expression of the Botulinum Toxin Type A-L (BoNt/A-L) Chain Fusion Proteins

This example describes an exemplary method to verify expression of the wild-type L chains, which may serve as a toxin, in bacteria harboring the pCA-L plasmids. Well isolated bacterial colonies harboring either pCAL are used to inoculate L-broth containing 0.1 mg/ml ampicillin and 2% (w/v) glucose, and grown overnight with shaking at 30° C. The overnight cultures are diluted 1:10 into fresh L-broth containing 0.1 mg/ml of ampicillin and incubated for 2 hours. Fusion protein expression is induced by addition of IPTG to a final concentration of 0.1 mM. After an additional 4 hour incubation at 30° C., bacteria are collected by centrifugation at 6,000×g for 10 minutes.

A small-scale SDS-PAGE analysis confirmed the presence of a 90 kDa protein band in samples derived from IPTG-induced bacteria. This Mr is consistent with the predicted size of a fusion protein having MBP (˜40 kDa) and BoNT/A-L chain (−50 kDa) components. Furthermore, when compared with samples isolated from control cultures, the IPTG-induced clones contained substantially larger amounts of the fusion protein.

The presence of the desired fusion proteins in IPTG-induced bacterial extracts is also confirmed by western blotting using the polyclonal anti-L chain probe described by Cenci di Bello et al., in Eur. J. Biochem. 219, 161 (1993). Reactive bands on PVDF membranes (Pharmacia; Milton Keynes, UK) are visualized using an anti-rabbit immunoglobulin conjugated to horseradish peroxidase (BioRad; Hemel Hempstead, UK) and the ECL detection system (Amersham, UK). Western blotting results confirmed the presence of the dominant fusion protein together with several faint bands corresponding to proteins of lower Mr than the fully sized fusion protein. This observation suggested that limited degradation of the fusion protein occurred in the bacteria or during the isolation procedure. Neither the use of 1 mM nor 10 mM benzamidine (Sigma; Poole, UK) during the isolation procedure eliminated this proteolytic breakdown.

The yield of intact fusion protein isolated by the above procedure remains fully adequate for all procedures described herein. Based on estimates from stained SDS-PAGE gels, the bacterial clones induced with IPTG yields 5-10 mg of total MBP-wild-type or mutant L chain fusion protein per liter of culture. Thus, the method of producing BoNT/A-L chain fusion proteins disclosed herein is highly efficient, despite any limited proteolysis that may occur.

The MBP-L chain fusion proteins encoded by the pCAL and pCAL-TyrU7 expression plasmids are purified from bacteria by amylose affinity chromatography. Recombinant wild-type or mutant L chains are then separated from the sugar binding domains of the fusion proteins by sitespecific cleavage with Factor X₂. This cleavage procedure yields free MBP, free L chains and a small amount of uncleaved fusion protein. While the resulting L chains present in such mixtures have been shown to possess the desired activities, additional purification step may be employed. Accordingly, the mixture of cleavage products is applied to a second amylose affinity column that bound both the MBP and uncleaved fusion protein. Free L chains are not retained on the affinity column, and are isolated for use in experiments described below.

In some embodiments, compounds of the present invention may be synthesized using techniques similar to the ones presented here. For example, a compound of the present invention comprising a light chain linked to a translocator may be synthesized using techniques similar to the ones presented here.

### EXAMPLE 3

### Purification of Fusion Proteins and Isolation of Recombinant BoNT/A-L Chains

This example describes a method to produce and purify wild-type recombinant BoNT/A light chains from bacterial clones. Pellets from 1 liter cultures of bacteria expressing the wild-type BoNT/A-L chain proteins are resuspended in column buffer [10 mM Tris-HCl (pH 8.0), 200 mM NaCl, 1 mM EGTA and 1 mM DTT] containing 1 mM phenylmethanesulfonyl fluoride (PMSF) and 10 mM benzamidine, and lysed by sonication. The lysates are cleared by centrifugation at 15,000×g for 15 minutes at 4° C. Supernatants are applied to an amylose affinity column [2×10 cm, 30 ml resin] (New England BioLabs; Hitchin, UK). Unbound proteins are washed from the resin with column buffer until the eluate is free of protein as judged by a stable absorbance reading at 280 nm. The bound MBP-L chain fusion protein is subsequently eluted with column buffer containing 10 mM maltose. Fractions containing the fusion protein are pooled and dialyzed against 20 mM Tris-HCl (pH 8.0) supplemented with 150 mM NaCl, 2 mM, CaCl2 and 1 mM DTT for 72 hours at 4° C.

Fusion proteins may be cleaved with Factor X₂ (Promega; Southampton, UK) at an enzyme: substrate ratio of 1:100 while dialyzing against a buffer of 20 mM Tris-HCl (pH 8.0) supplemented with 150 mM NaCl, 2 mM, CaCl₂ and 1 mM DTT. Dialysis is carried out for 24 hours at 4° C. The mixture of MBP and either wild-type or mutant L chain that resulted from the cleavage step is loaded onto a 10 ml amylose column equilibrated with column buffer. Aliquots of the flow through fractions are prepared for SDS-PAGE analysis to identify samples containing the L chains. Remaining portions of the flow through fractions are stored at −20° C. Total E. coli extract or the purified proteins are solublized in SDS sample buffer and subjected to PAGE according to standard procedures. Results of this procedure indicate the recombinant toxin fragment accounted for roughly 90% of the protein content of the sample.

The foregoing results indicate that the approach to creating MBP-L chain fusion proteins described herein may be used to efficiently produce wild-type and mutant recombinant BoNT/A-L chains. Further, the results demonstrate that recombinant L chains may be separated from the maltose binding domains of the fusion proteins and purified thereafter.

A sensitive antibody-based assay is developed to compare the enzymatic activities of recombinant L chain products and their native counterparts. The assay employed an antibody having specificity for the intact C-terminal region of SNAP-25 that corresponded to the BoNT/A cleavage site. Western Blotting of the reaction products of BoNT/A cleavage of SNAP-25 indicated an inability of the antibody to bind SNAP-25 sub-fragments. Thus, the antibody recompound employed in the following Example detected only intact SNAP-25. The loss of antibody binding served as an indicator of SNAP-25 proteolysis mediated by added BoNT/A light chain or recombinant derivatives thereof.

### EXAMPLE 5

### Evaluation of the Proteolytic Activities of Recombinant L Chains Against a SNAP-25 Substrate

Both native and recombinant BoNT/A-L chains can proteolyze a SNAP-25 substrate. A quantitative assay may be employed to compare the abilities of the wild-type and their recombinant analogs to cleave a SNAP-25 substrate.

The substrate utilized for this assay is obtained by preparing a glutathione-S-transferase (GST)-SNAP-25 fusion protein, containing a cleavage site for thrombin, expressed using the pGEX-2T vector and purified by affinity chromatography on glutathione agarose. The SNAP-25 is then cleaved from the fusion protein using thrombin in 50 mM Tris-HCl (pH 7.5) containing 150 mM NaCl and 2.5 mM CaCl₂ (Smith et al. Gene 67, 31 (1988) at an enzyme:substrate ratio of 1:100. Uncleaved fusion protein and the cleaved glutathione-binding domain bound to the gel. The recombinant SNAP-25 protein is eluted with the latter buffer and dialyzed against 100 mM HEPES (pH 7.5) for 24 hours at 4° C. The total protein concentration is determined by routine methods.

Rabbit polyclonal antibodies specific for the C-terminal region of SNAP-25 are raised against a synthetic peptide having the amino acid sequence, CANQRATKMLGSG (SEQ ID NO: 35). This peptide corresponds to residues 195 to 206 of the synaptic plasma membrane protein and an N-terminal cysteine residue not found in native SNAP-25. The synthetic peptide is conjugated to bovine serum albumin (BSA) (Sigma; Poole, UK) using maleimidobenzoyl-N-hydroxysuccinimide ester (MBS) as a cross-linking compound (Sigma; Poole, UK) to improve antigenicity (Liu et al., Biochemistry 18, 690 (1979). Affinity purification of the anti-peptide antibodies is carried out using a column having the antigenic peptide conjugated via its N-terminal cysteine residue to an aminoalkyl agarose resin (Bio-Rad; Hemel Hempstead, UK), activated with iodoacetic acid using the cross-linker ethyl 3-(3-dimethytpropyl) carbodiimide. After successive washes of the column with a buffer containing 25 mM Tris-HCl (pH 7.4) and 150 mM NaCl, the peptide-specific antibodies are eluted using a solution of 100 mM glycine (pH 2.5) and 200 mM NaCl, and collected in tubes containing 0.2 ml of 1 M Tris-HCl (pH 8.0) neutralizing buffer.

All recombinant preparations containing wild-type L chain are dialyzed overnight at 4° C. into 100 mM HEPES (pH 7.5) containing 0.02% Lubrol and 10 μM zinc acetate before assessing their enzymatic activities. BoNT/A, previously reduced with 20 mM DTT for 30 minutes at 37° C., as well as these dialyzed samples, are then diluted to different concentrations in the latter HEPES buffer supplemented with 1 mM DTT.

Reaction mixtures include 5 μl recombinant SNAP-25 substrate (8.5 μM final concentration) and either 20 μl reduced BoNT/A or recombinant wild-type L chain. All samples are incubated at 37° C. for 1 hour before quenching the reactions with 25 μl aqueous 2% trifluoroacetic acid (TFA) and 5 mM EDTA, Foran et al. (1994, Biochemistry 33, 15365). Aliquots of each sample are prepared for SDS-PAGE and Western blotting with the polyclonal SNAP-25 antibody by adding SDS-PAGE sample buffer and boiling. Anti-SNAP-25 antibody reactivity is monitored using an ECL detection system and quantified by densitometric scanning.

Western blotting results indicate clear differences between the proteolytic activities of the purified mutant L chain and either native or recombinant wild-type BoNT/A-L chain. Specifically, recombinant wild-type L chain cleaves the SNAP-25 substrate, though somewhat less efficiently than the reduced BoNT/A native L chain that serves as the positive control in the procedure. Thus, an enzymatically active form of the BoNT/A-L chain is produced by recombinant means and subsequently isolated. Moreover, substitution of a single amino acid in the L chain protein abrogated the ability of the recombinant protein to degrade the synaptic terminal protein.

As a preliminary test of the biological activity of the wild-type recombinant BoNT/A-L chain, the ability of the MBP-L chain fusion protein to diminish Ca²⁺-evoked catecholamine release from digitonin-permeabilized bovine adrenochromaffin cells is examined. Consistently, wild-type recombinant L chain fusion protein, either intact or cleaved with Factor X₂ to produce a mixture containing free MBP and recombinant L chain, induced a dose-dependent inhibition of Ca²⁺-stimulated release equivalent to the inhibition caused by native BoNT/A.

### EXAMPLE 6

### Method of Treating a Neuromuscular Disorder: Treatment of Spasmodic Torticollis

A male, age 45, suffering from spasmodic torticollis, as manifested by spasmodic or tonic contractions of the neck musculature, producing stereotyped abnormal deviations of the head, the chin being rotated to the side, and the shoulder being elevated toward the side at which the head is rotated, is treated by injection with about 8 U/kg to about 15 U/kg of neurotoxins of the present invention (e.g., a botulinum toxin type A linked to a translocator comprising a human immunodeficiency virus transactivator protein peptide, SEQ ID NO: 5). After 3-7 days, the symptoms are substantially alleviated; i.e., the patient is able to hold his head and shoulder in a normal position. The alleviation persists for about 7 months to about 27 months.

### EXAMPLE 7

### Method of Treating Pain

A) Treatment of Pain Associated with Muscle Disorder

An unfortunate 36 year old woman has a 15 year history of temporomandibular joint disease and chronic pain along the masseter and temporalis muscles. Fifteen years prior to evaluation she noted increased immobility of the jaw associated with pain and jaw opening and closing and tenderness along each side of her face. The left side is originally thought to be worse than the right. She is diagnosed as having temporomandibular joint (TMJ) dysfunction with subluxation of the joint and is treated with surgical orthoplasty meniscusectomy and condyle resection.

She continues to have difficulty with opening and closing her jaw after the surgical procedures and for this reason, several years later, a surgical procedure to replace prosthetic joints on both sides is performed. After the surgical procedure progressive spasms and deviation of the jaw ensues. Further surgical revision is performed subsequent to the original operation to correct prosthetic joint loosening. The jaw continues to exhibit considerable pain and immobility after these surgical procedures. The TMJ remained tender as well as the muscle itself. There are tender points over the temporomandibular joint as well as increased tone in the entire muscle. She is diagnosed as having post-surgical myofascial pain syndrome and is injected with about 8 U/kg to about 15 U/kg of the modified neurotoxin (e.g., a botulinum toxin type A linked to a translocator comprising a human immunodeficiency virus transactivator protein peptide (SEQ ID NO: 5) into the masseter and temporalis muscles.

Several days after the injections she noted substantial improvement in her pain and reports that her jaw feels looser. This gradually improves over a 2 to 3 week period in which she notes increased ability to open the jaw and diminishing pain. The patient states that the pain is better than at any time in the last 4 years. The improved condition persists for up to 27 months after the original injection of the modified neurotoxin.

(B) Treatment of Pain Subsequent to Spinal Cord Injury

A patient, age 39, experiencing pain subsequent to spinal cord injury is treated by intrathecal administration, for example by spinal tap or by catherization (for infusion), to the spinal cord, with about 0.1 U/kg to about 10 U/kg of the modified neurotoxin (e.g., a botulinum toxin type A linked to a translocator comprising a human immunodeficiency virus transactivator protein peptide, SEQ ID NO: 5). The particular toxin dose and site of injection, as well as the frequency of toxin administrations depend upon a variety of factors within the skill of the treating physician, as previously set forth. Within about 1 to about 7 days after the modified neurotoxin administration, the patient's pain is substantially reduced. The pain alleviation persists for up to 27 months.

### EXAMPLE 8

### Method of Treating an Autonomic Disorder: Treatment of Excessive Sweating

A male, age 65, with excessive unilateral sweating is treated by administering 0.05 U/kg to about 2 U/kg of a modified neurotoxin, depending upon degree of desired effect. An example of a modified neurotoxin include a botulinum toxin type A linked to a translocator comprising a human immunodeficiency virus transactivator protein peptide (SEQ ID NO: 5) The administration is to the gland nerve plexus, ganglion, spinal cord or central nervous system. The specific site of administration is to be determined by the physician's knowledge of the anatomy and physiology of the target glands and secretary cells. In addition, the appropriate spinal cord level or brain area can be injected with the toxin. The cessation of excessive sweating after the modified neurotoxin treatment is up to 27 months.

Various articles and patents have been cited here. The disclosures of these references are incorporated in their entirety herein by reference herein. Other references in which the disclosures are incorporated in their entirety by reference herein include: Kabouridis P. Biological applications of protein transduction technology, Trends in Biotechnology, Vol 21 No 11 Nov. 2003; Morris et al., Translocating peptides and proteins and their use for gene delivery, Current Opinion in Biotechnology 2000, 11:461-466; Fernandez-Salas et al., Is the light chain subcellular localization an important factor in botulinum toxin duration of action?, Movement Disorders, Vol 19 Supp 8, 2004, pp. S23-S24; Fernandez-Salas et al., Plasma membrane localization signals in the light chain of botulinum toxin, PNAS, March 2004, Vol 101 No 9; Will et al., Unmodified Cre recombinase crosses the membrane, Nucleic Acids Research, 2002 Vol 30 No 12 e59; Pepperl-Klindworth et al., Gene Therapy 2003, Vol 10, 278-284; Langedijk et al., Molecular Diversity, Vol 8, 101-111 2004; Noguchi et al., PDX-1 protein containing its own Antennapedia-like protein transduction domain can transduce pancreatic duct and islet cells, Diabetes, Vol 52, 1732-1737, 2003.

While this invention has been described with respect to various specific examples and embodiments, it is to be understood that the invention is not limited thereto and that it can be variously practiced with the scope of the following claims.

What is claimed is:

1. A compound comprising a toxin linked to a translocator that comprises a protein transduction domain.

2. The compound of claim 1 wherein more than one toxin is linked to the translocator.

3. The compound of claim 1 wherein the toxin is linked to more than one translocator.

4. The compound of claim 1 wherein the toxin comprises a light chain of a botulinum toxin type A, B, C₁, D, E, F, G or mixtures thereof.

5. The compound of claim 1 wherein the toxin comprises a light chain of botulinum toxin type A.

6. The compound of claim 1 wherein the toxin comprises (i) a light chain of a botulinum toxin type A, B, C₁, D, E, F or G, and (ii) a heavy chain of a botulinum toxin type A, B, C₁, D, E, F, G, or parts thereof.

7. The compound of claim 1 wherein the translocator is a ciliary neurotrophic factor, caveolin, interleukin 1 beta, thioredoxin, fibroblast growth factor-1, fibroblast growth factor-2, Human beta-3, integrin, lactoferrin, Engrailed, Hoxa-5, Hoxb-4 or Hoxc-8.

8. The compound of claim 1 wherein the translocator comprises penetratin peptide, Kaposi fibroblast growth factor membrane-translocating sequence, nuclear localization signal, transportan, herpes simplex virus type 1 protein 22, human immunodeficiency virus transactivator protein or combinations thereof.

9. The compound of claim 1 wherein the translocator comprises a peptide selected from the group consisting of a Kaposi fibroblast growth factor membrane-translocating sequence (SEQ ID NO: 1), nuclear localization signal (SEQ ID NO: 2), transportan (SEQ ID NO: 3), herpes simplex virus type 1 protein 22 (SEQ ID NO: 4) and human immunodeficiency virus transactivator protein peptide (SEQ ID NO: 5).

10. The compound of claim 1 wherein the translocator comprises a human immunodeficiency virus transactivator protein peptide (SEQ ID NO: 5).

11. The compound of claim 1 wherein the translocator comprises a penetratin peptide selected from the group consisting of SEQ ID NO: 6, SEQ ID NO: 7, SEQ ID NO: 8, SEQ ID NO: 9, SEQ ID NO: 10; SEQ ID NO: 11, SEQ ID NO: 12, SEQ ID NO: 13, SEQ ID NO: 14, SEQ ID NO: 15 and SEQ ID NO: 16.

12. The compound of claim 1 wherein the toxin comprises a light chain of botulinum toxin, and the translocator comprises a human immunodeficiency virus transactivator protein peptide (SEQ ID NO: 5).

13. The compound of claim 1 further comprising a protease cleavage domain.

14. The compound of claim 13 wherein the protease cleavage domain is a substrate for a blood protease.

15. The compound of claim 14 wherein the blood protease is a thrombin, coagulation factor Xa, coagulation factor XIa, coagulation factor XIIa, coagulation factor IXa, coagulation factor VIIa, kallikrein, protein C, MBP-associated serine protease, oxytocinase, ADAM-TS13 or lysine carboxypeptidase.

16. The compound of claim 1 further comprising a protease cleavage domain, wherein the toxin comprises a light chain of botulinum toxin type, the translocator comprises a human immunodeficiency virus transactivator protein peptide (SEQ ID NO: 5) and the protease cleavage domain is a substrate for a thrombin.

17. The compound of any of claims 1-16 further comprising a targeting moiety.

18. A compound comprising a toxin linked to a translocator, the toxin comprises a light chain of a botulinum toxin type A, and the translocator comprises a human immunodeficiency virus transactivator protein peptide (SEQ ID NO: 5).

19. The compound of claim 18 wherein the compound further comprises a targeting moiety.

20. The compound of claim 18 wherein the compound further comprises a protease cleavage domain.

21. A method of translocating a compound comprising a toxin across a cell membrane, the method comprises linking the toxin to a translocator that comprises a protein transduction domain.

22. A method of treating a biological disorder in a patient, the method comprises locally administering a compound of claim 1 to a patient in need thereof.

23. The method of claim 22 wherein the biological disorder comprises at least one of a neuromuscular disorder, an autonomic disorder and pain.

24. The method of treating a biological disorder of claim 23, wherein the treatment of the neuromuscular disorder comprises the step of locally administering a compound of claim 1 to a group of muscles.

25. The method of treating a biological disorder of claim 23, wherein the treatment of the autonomic disorder comprises the step of locally administering a compound of claim 1 to a gland.

26. The method of treating a biological disorder claim 23, wherein the treatment of pain comprises the step of administering a compound of claim 1 to a site of pain.

27. The method of treating a biological disorder of claim 23, wherein the treatment of pain comprises the step of administering a compound of claim 1 to a spinal cord.


SEQUENCE LISTING (34 sequences)

SEQ ID NO: 1 - protein, 16 aa, Artificial Sequence
  PDT of Kaposi-fibroblast growth factor membrane-translocating sequence (kFGF MTS)
AAVALLPAVLLALLAP

SEQ ID NO: 2 - protein, 12 aa, Artificial Sequence
  PDT of the nuclear localization signal (NLS)
TPPKKKRKVEDP

SEQ ID NO: 3 - protein, 27 aa, Artificial Sequence
  PDT of Transportan
GWTLNSAGYLLGKINLKALAALAKKIL

SEQ ID NO: 4 - protein, 34 aa, Artificial Sequence
  PDT of herpes simplex virus type 1 protein 22 (VP22)
DAATATRGRSAASRPTERPRAPARSASRPRRPVE

SEQ ID NO: 5 - protein, 11 aa, Artificial Sequence
  PDT of human immunodeficiency virus transactivator protein (TAT, 47-57)
YGRKKRRQRRR

SEQ ID NO: 6 - protein, 16 aa, Artificial Sequence
  Penetratin peptide
RQIKIWFQNRRMKWKK

SEQ ID NO: 7 - protein, 16 aa, Artificial Sequence
  Penetratin peptide
KKWKMRRNQFWIKIQR

SEQ ID NO: 8 - protein, 16 aa, Artificial Sequence
  Penetratin peptide
RQIKIWFQNRRMKWKK

SEQ ID NO: 9 - protein, 16 aa, Artificial Sequence
  Penetratin peptide
RQIKIWFPNRRMKWKK

SEQ ID NO: 10 - protein, 16 aa, Artificial Sequence
  Penetratin peptide
RQPKIWFPNRRMPWKK

SEQ ID NO: 11 - protein, 16 aa, Artificial Sequence
  Penetratin peptide
RQIKIWFQNMRRKWKK

SEQ ID NO: 12 - protein, 16 aa, Artificial Sequence
  Penetratin peptide
RQIRIWFQNRRMRWRR

SEQ ID NO: 13 - protein, 16 aa, Artificial Sequence
  Penetratin peptide
RRWRRWWRRWWRRWRR

SEQ ID NO: 14 - protein, 16 aa, Artificial Sequence
  Penetratin peptide
RQIKIFFQNRRMKFKK

SEQ ID NO: 15 - protein, 15 aa, Artificial Sequence
  Penetratin peptide
TERQIKIWFQNRRMK

SEQ ID NO: 16 - protein, 15 aa, Artificial Sequence
  Penetratin peptide
KIWFQNRRMKWKKEN

SEQ ID NO: 17 - protein, 438 aa, Artificial Sequence
  Amino acid sequence of LC
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 18 - protein, 849 aa, Artificial Sequence
  Amino acid sequence of HC
ALDNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 19 - protein, 440 aa, Artificial Sequence
  Amino acid sequence of LC
PVTINNFNYNDPIDNDNIIMMEPPFARGTGRYYKAFKITDRIWIIPERYTFGYKPEDFNKSSGIFNRDVCEYYDPDYLNTNDKKNIFFQTLIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDTIQAEELYTFGGQDPSIISPSTDKSIYDKVLQNFRGIVDRLNKVLVCISDPNININIYKNKFKDKYKFVEDSEGKYSIDVESFNKLYKSLMLGFTEINIAENYKIKTRASYFSDSLPPVKIKNLLDNEIYTIEEGFNISDKNMGKEYRGQNKAINKQAYEEISKEHLAVYKIQMCKSVK

SEQ ID NO: 20 - protein, 900 aa, Artificial Sequence
  Amino acid sequence of HC
YTIEEGFNISDKNMGKEYRGQNKAINKQAYEEISKEHLAVYKIQMCKSVKVPGICIDVDNENLFFIADKNSFSDDLSKNERVEYNTQNNYIGNDFPINELILDTDLISKIELPSENTESLTDFNVDVPVYEKQPAIKKVFTDENTIFQYLYSQTFPLNIRDISLTSSFDDALLVSSKVYSFFSMDYIKTANKVVEAGLFAGWVKQIVDDFVIEANKSSTMDKIADISLIVPYIGLALNVGDETAKGNFESAFEIAGSSILLEFIPELLIPVVGVFLLESYIDNKNKIIKTIDNALTKRVEKWIDMYGLIVAQWLSTVNTQFYTIKEGMYKALNYQAQALEEIIKYKYNIYSEEEKSNININFNDINSKLNDGINQAMDNINDFINECSVSYLMKKMIPLAVKKLLDFDNTLKKNLLNYIDENKLYLIGSVEDEKSKVDKYLKTIIPFDLSTYSNIEILIKIFNKYNSEILNNIILNLRYRDNNLIDLSGYGAKVEVYDGVKLNDKNQFKLTSSADSKIRVTQNQNIIFNSMFLDFSVSFWIRIPKYRNDDIQNYIHNEYTIINCMKNNSGWKISIRGNRIIWTLIDINGKTKSVFFEYNIREDISEYINRWFFVTITNNLDNAKIYINGTLESNMDIKDIGEVIVNGEITFKLDGDVDRTQFIWMKYFSIFNTQLNQSNIKEIYKIQSYSEYLKDFWGNPLMYNKEYYMFNAGNKNSYIKLVKDSSVGEILIRSKYNQNSNYINYRNLYIGEKFIIRRESNSQSINDDIVRKEDYIHLDLVLHHEEWRVYAYKYFKEQEEKLFLSIISDSNEFYKTIEIKEYDEQPSYSCQLLFKKDEESTDDIGLIGIHRFYESGVLRKKYKDYFCISKWYLKEVKRKPYKSNLGCNWQFIPKDEGWTE

SEQ ID NO: 21 - protein, 448 aa, Artificial Sequence
  Amino acid seqence of LC
PITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNK

SEQ ID NO: 22 - protein, 842 aa, Artificial sequence
  Amino acid sequence of HC
TLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 23 - protein, 442 aa, Artificial Sequence
  Amino acid sequence of LC
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 24 - protein, 834 aa, Artificial Sequence
  Amino acid seqence of HC
NSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 25 - protein, 422 aa, Artificial Sequence
  Amino acid sequence of LC
PKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRK

SEQ ID NO: 26 - protein, 829 aa, Artificial Sequence
  Amino acid sequence of HC
SICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 27 - protein, 436 aa, Artificial Sequence
  Amino acid sequence of LC
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRK

SEQ ID NO: 28 - protein, 838 aa, Artificial Sequence
  Amino acid sequence of HC
GTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 29 - protein, 441 aa, Artificial Sequence
  Amino acid sequence of LC
PVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYK

SEQ ID NO: 30 - protein, 855 aa, Artificial Sequence
  Amino acid sequence of HC
NTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 31 - protein, 15 aa, Artificial Sequence
  Amino acid sequence of an immunoglobulin g1 hinge region
EPKSCDKTHTCPPCP

SEQ ID NO: 32 - DNA, 23 bp, Artificial Sequence
  Primer of Clostridium botulinum type A light chain
aaaggccttttgttaataaacaa

SEQ ID NO: 33 - DNA, 26 bp, Artificial Sequence
  Primer of Clostridium botulinum type A light chain
ggaattcttacttattgtatccttta

SEQ ID NO: 34 - protein, 13 aa, Artificial Sequence
  Amino acid sequence of the C-terminal region of SNAP-25
CANQRATKMLGSG